(19) United States
(12) Patent Application Publication
(10) Pub. No.: US 2020/0038217 A1
(43) Pub. Date: Feb. 6, 2020

(54) Manual Therapist Support Apparatus

(71) Applicants: Manvir Singh Purewal, Surrey (CA); Simon Kelly, Vancouver (CA)

(72) Inventors: Manvir Singh Purewal, Surrey (CA); Simon Kelly, Vancouver (CA)

(21) Appl. No.: 16/052,344

(22) Filed: Aug. 1, 2018

(51) Int. Cl. A61F 5/01; A61F 5/37

(52) U.S. Cl. CPC A61F 5/0118; A61F 5/37

(57) ABSTRACT

A support apparatus for use with manual therapist. The support apparatus having a forearm support and an upper arm support. The forearm support has a forearm interlock end and a wrist end. The upper arm support has an upper arm interlock end that is configured to interlock with forearm interlock end when the arm is extended. The upper arm support has an upper arm support end configured to be placed in the underarm of the user. Forearm and upper arm attachments couples to the support apparatus to temporarily attach the support apparatus to the user's arm, wrist and hand.

### REFERENCE TO PENDING APPLICATIONS

This application does not claim the benefit of any issued U.S. Patent or pending application.

### BACKGROUND OF THE INVENTION

### Field of the Invention

The present invention generally relates to manual therapist support device, and in particular to a manual therapist support device that provides support to the arm, wrist and thumb of a manual therapist.

### Background

The present invention relates to a manual therapist support apparatus, and, in particular, to an apparatus for immobilizing the thumb, wrist and elbow to provide additional strength and support to the manual therapist's body. In this application, the term manual therapist refers to individual who manually provide therapy, including but not limited to massage therapists, physiotherapists and physical therapist.

The physical demands of massage therapy are many. Manual therapists often injure their thumb, i.e. the thumb carpometacarpal (CMC) joint, along with their wrist and elbow joints due to a combination of overuse and poor body mechanics. Further, a manual therapist may suffer tenosynovitis, carpal tunnel syndrome and other medical conditions.

It would therefore be a significant advance in the art, and is accordingly an object of the present invention, to reduce the number of injuries to manual therapists. Thus, there is a need for an apparatus that provides support to a manual therapist's arm, wrist and thumb.

### BRIEF SUMMARY OF THE INVENTION

The present invention generally relates to manual therapist support device, and in particular to a manual therapist support device that provides support to the arm, wrist and thumb of a manual therapist.

In one aspect, a manual therapist support apparatus is disclosed. The support apparatus includes a forearm support and an upper arm support. The forearm support has a wrist end and a forearm interlock end. The upper arm support has an upper arm interlock end and a support end. The upper arm interlock end is configured to interlock with the forearm interlock end when the user's arm is extended, while the support end configured to be placed in the underarm of the user. The support apparatus further includes an apparatus attachment that couples to the forearm support and the upper arm support to temporarily attach the support apparatus to the user's forearm, wrist upper arm.

In one aspect of the support apparatus, the upper arm interlock end is a male interlock member while the forearm interlock end is a female interlock member. The female interlock member is configured to receive the male interlock member when the user's arm is extended.

In another aspect, the support apparatus further includes an elbow support attachment that couples to the forearm support and to the upper arm support proximate the user's elbow.

In another aspect, the support apparatus further includes a thumb support configured to receive a thumb of a user. The thumb support may be detachably connected to the wrist end of the forearm support.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention wherein similar characters of reference denote corresponding parts in each view,

FIG. 1 is a perspective side view of an embodiment of the present invention illustrating embodiments of the forearm support and upper arm support.

FIG. 2 is a perspective side view of an embodiment of the present invention illustrating embodiments of the upper arm support and forearm support in a bent position.

FIG. 3 is a perspective side view of an embodiment of the present invention illustrating embodiments of the upper arm and forearm supports.

FIG. 4 is a perspective view of an embodiment of the present invention illustrating embodiments of the thumb support.

FIG. 5 is a perspective view of an embodiment of the thumb support of the present invention.

### DETAILED DESCRIPTION OF THE INVENTION

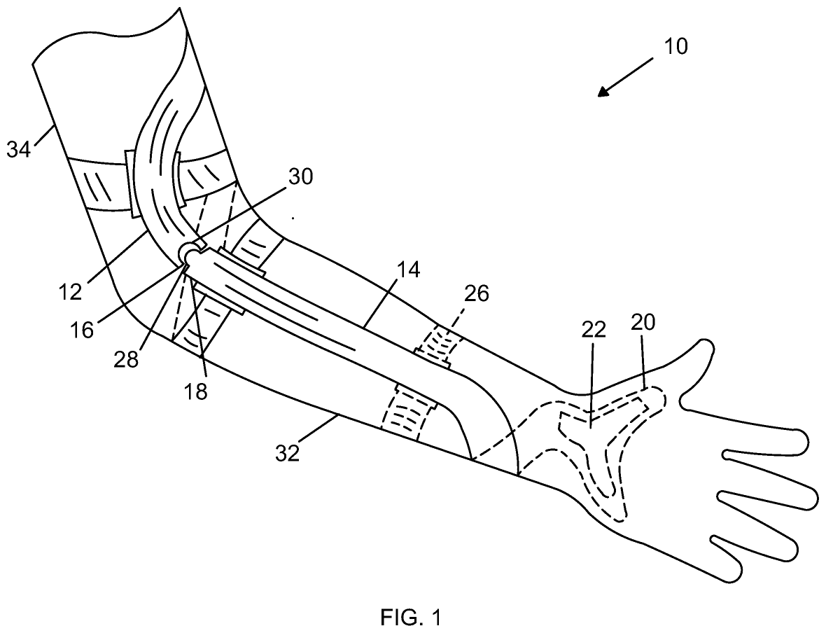

As illustrated in FIGS. 1-3, an embodiment of a manual therapist support apparatus 10 is illustrated. Support apparatus 10 includes a forearm support 14 and an upper arm support 12. Support apparatus 10 is configured to reduce the force and pressure to the manual therapists elbow, wrist and finger joints, including the thumb carpometacarpal (CMC) joint. This reduction in the force and pressure, reduces the likelihood for injury to the manual therapist which in turn allows the manual therapist to provide quality manual therapy services while maintaining a more healthy and safe body.

Forearm support 14 is configured to be located along the forearm 32 of a user. In some embodiments, forearm support 14 is located along the ulna side of forearm 32. Forearm support 14 may be constructed from rigid material such as plastic, metal or a combination thereof. Those skilled in the art will recognize various materials may be utilized and are within the scope of the present invention.

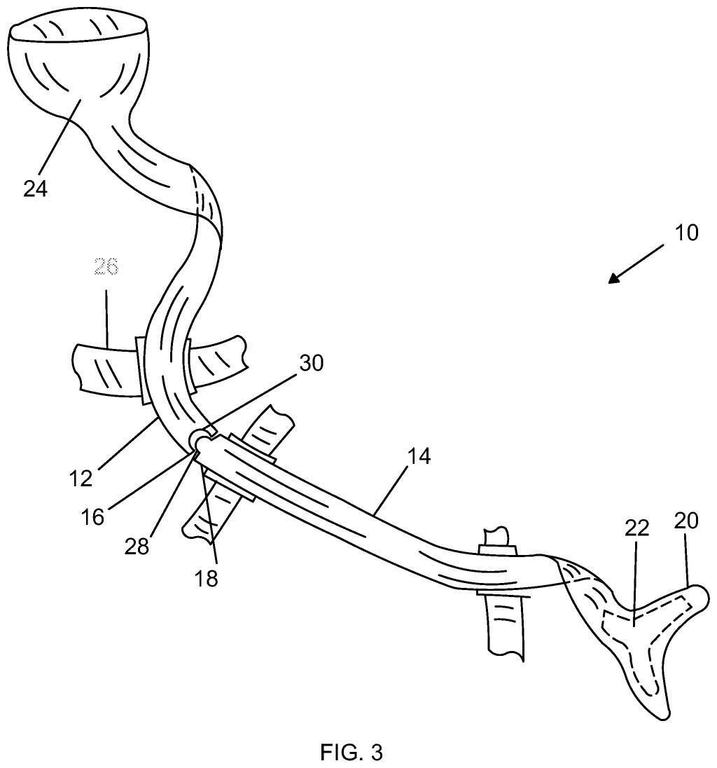

Forearm support 14 has a wrist end 20 and a forearm interlock end 18. Forearm wrist end 20 may have a pressure pad 22 located along the palm of the user. Pressure pad 22 may be constructed from a flexible material that can allow for flexibility due to the movement of the wrist and hand, see FIG. 3. Those skilled in the art will recognize that various materials such as plastic or metal may be utilized and are within the scope of the present invention.

Upper arm support 12 is configured to be located along the upper arm 34 of a user. Upper arm support 12 may be constructed from rigid material such as plastic, metal or a combination thereof. Those skilled in the art will recognize various materials may be utilized and are within the scope of the present invention.

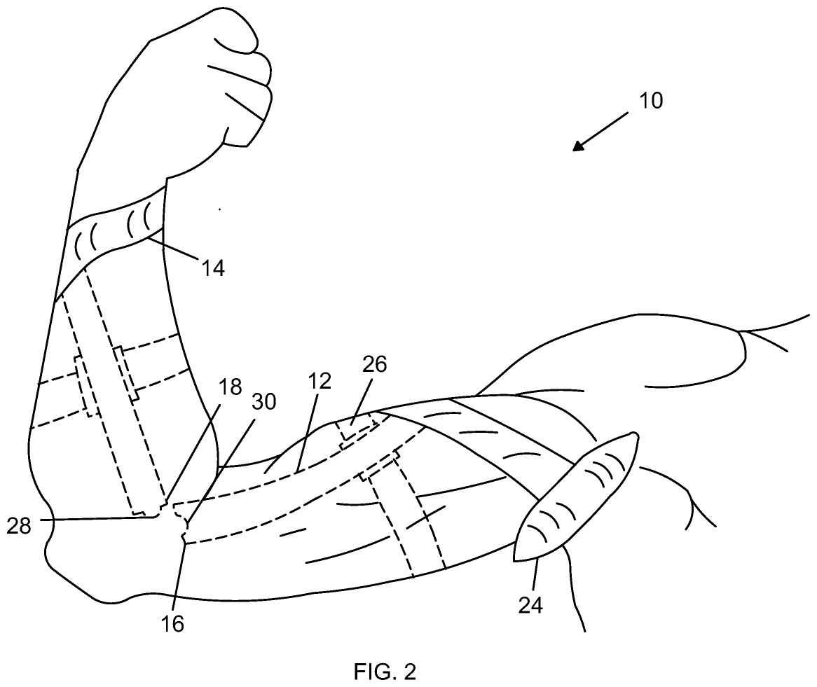

Upper arm support 12 has an upper arm interlock end 16 and a support end 44, see FIG. 2. The upper arm interlock end 16 is configured to interlock with the forearm interlock end 18 when the user's arm is extended.

In this embodiment, the interlock is a male/female interlock mechanism where forearm interlock end 18 is configured to be the male interlock member 28 and the upper arm interlock end 16 is configured to be a female interlock member 30 configured to receive the male interlock member 28 when the user's arm is extended, see FIG. 3.

Support end 24 is configured to be similar to a crutch pad and is placed in the underarm of the user. Support attachment 26 may be coupled to upper arm support 12 and forearm support 14 to temporarily attach upper arm support 12 and forearm support 14 to the user's arm. Support attachment 26 may be constructed from hook and mesh material, or any other type of material that allows for the temporary attachment of apparatus 10 to the users arm wrist and hand.

The attachment of the support apparatus 10 to the user's arm and the placement of support end 24 in the underarm of the user assists the user to be able to exert force through the body into the hand allowing for more forceful manual manipulation without over exerting the hands, wrist and fingers when the upper arm support 12 and the forearm support 14 are locked together through the interlock mechanism. This locking capability allows for pressure and force on the hand, wrist and forearm of a user to be transferred to the upper torso of the user. The reduction of pressure and force on the hand, wrist and forearm allows the user to provide the desired manual therapy while avoiding detrimental forces thereon.

By having female interlock member 30 configured to receive the male interlock member 28 when the user's arm is extended allows for the freedom of movement of the user's arm during a therapy session. As is illustrated in FIG. 2, when the user's arm is bent, the female interlock member 30 and the male interlock member 28 are not engaged. This allows the user's arm to be free. Once the user, however, extends the arm, the female interlock member 30 and the male interlock member 28 engage to allow for the force transfer.

In an additional embodiment of the present invention, the support apparatus 10 may also include an elbow attachment the couples to the forearm support and the upper arm support temporarily attach upper arm support 12 and forearm support 14 around the user's elbow. Elbow attachment may be constructed from hook and mesh material, or any other type of material that allows for the temporary attachment of apparatus 10 around the user's elbow.

An embodiment of forearm wrist end 70 is illustrated in FIG. 5. In this embodiment, forearm wrist end 70 includes a wrist attachment 58 the couples to forearm wrist end 70 of forearm support 72 to temporarily attach forearm wrist end 70 to the user's wrist. A pressure pad 76 located along the palm of the user. Pressure pad 76 may be constructed from a flexible material that can allow for flexibility due to the movement of the wrist and hand. Connected to pressure pad 76 is a leaf spring 74 which may provide a range of motion of the user's wrist while providing additional support and flexibility to pressure pad 76.

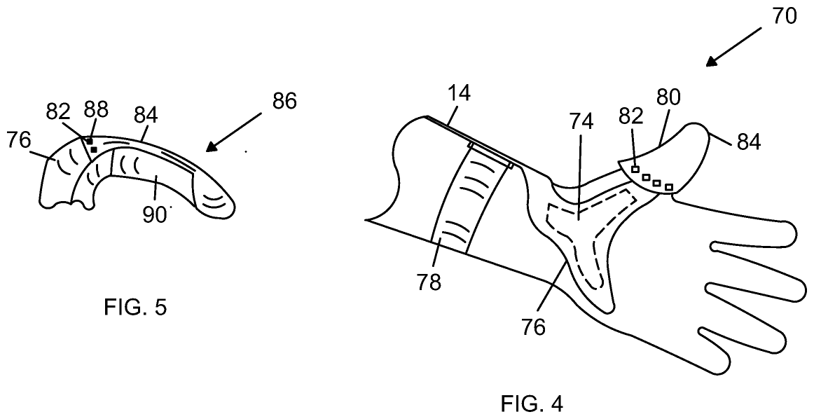

As illustrated in FIGS. 4 and 5, an additional embodiment of support apparatus 10 includes a thumb support 80. Thumb support 80 is configured to be located along the top and end of a user's thumb in order to provide support to the user's hand and thumb including the user's CMC joint. Thumb support 80 may include a rigid base support 82 connected to a flexible thumb attachment 84. Base support 82 may be constructed from a rigid material that can allow for flexibility due to the movement of the thumb. Thumb attachment 84 may be constructed from flexible material that can be secured over the tip end of the thumb. Those skilled in the art will recognize that various materials such as plastic metal and hook and mash material may be utilized and are within the scope of the present invention.

In this embodiment, thumb support 80 is secured to forearm wrist end 20 by a series of interlocking raised connecting points 86 located on forearm wrist end 20 and a matching series of interlocking openings 88 located along thumb support 80.

While preferred embodiments of the present inventive concept have been shown and disclosed herein, it will be obvious to those persons skilled in the art that such embodiments are presented by way of example only, and not as a limitation to the scope of the inventive concept. Variations, changes, and substitutions may occur or be suggested to those skilled in the art without departing from the intent, scope, and totality of this inventive concept. Such variations, changes, and substitutions may involve other features which are already known per se and which may be used instead of, in combination with, or in addition to features already disclosed herein. Accordingly, it is intended that this inventive concept be inclusive of such variations, changes, and substitutions, and by no means limited by the scope of the claims presented herein.

I claim:

1. A support apparatus, comprising:
a forearm support having a wrist end and a forearm interlock end;
an upper arm support having an upper arm interlock end and a support end, the upper arm interlock end configured to interlock with the forearm interlock end when the user's arm is extended, the support end configured to be placed in the underarm of the user;
and an apparatus attachment that couples to the forearm support and the upper arm support to temporarily attach the support apparatus to the user's forearm, wrist upper arm.

2. The support apparatus of claim 1 wherein upper arm interlock end is a male interlock member and the forearm interlock end is a female interlock member configured to receive the male interlock member when the user's arm is extended.

3. The support apparatus of claim 1, further comprising an elbow support attachment that couples to the forearm support and to the upper arm support proximate the user's elbow.

4. The support apparatus of claim 1, further comprising a thumb support configured to receive a thumb of a user, the thumb support detachably connected to the wrist end of the forearm support.

5. The support apparatus of claim 4, wherein the thumb support comprises a rigid base support connected to a flexible thumb attachment.
